(19) United States
(12) Patent Application Publication
(10) Pub. No.: US 2006/0020249 A1
(43) Pub. Date: Jan. 26, 2006

(54) Absorbent articles having wetness-indicating character graphics providing an interactive training aid

(75) Inventor: Melanie D. Allen, Milford, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/899,257

(22) Filed: Jul. 26, 2004

(51) Int. Cl. A61F 13/15

(52) U.S. Cl. 604/361

(57) ABSTRACT

An absorbent article having an outer cover which has an interior surface and an opposite exterior surface; an absorbent assembly disposed on the interior surface; a permanent character graphic disposed on the outer cover; a permanent object graphic disposed on the outer cover; wherein the permanent character graphic is interactively interrelated with the permanent object graphic; and further having at least one active character graphic, wherein said active character graphic is related in subject matter with said permanent character graphic and said permanent object graphic. The active character graphic may become significantly more visible or invisible when exposed to urine. Alternatively, the active character graphic may become significantly more visible or invisible with the passage of time when exposed to the environment but not exposed to urine.

### BACKGROUND OF THE INVENTION

In the later stages of baby development, an infant is trained to make the transition from diapers to training pants and ultimately to using a toilet. In providing such training, parents and caregivers (hereinafter collectively referred to as “caregiver”) find it difficult to communicate the overall process and need for toilet training to the infant. As a result, both the caregiver and infant become frustrated.

In an effort to assist in toilet training, diaper manufacturers now sell disposable absorbent articles having toilet training aids. One such example is depicted in U.S. Pat. No. 6,635,797, issued on Oct. 21, 2003 to Olson et al (hereinafter referred to as “Olson”) which illustrates an absorbent article having wetness indicating graphics that provide an interactive training aid. More specifically, referring now to FIG. 1 of the Olson patent, an absorbent article is shown having a permanent character graphic (i.e., dog having human-like expressions), a permanent object graphic (i.e., boat) and a plurality of active object graphics (i.e., fish without human-like expressions); such that, when an infant fails to use the toilet by urinating in his/her diaper, the presence of urine causes the fish to disappear. It is proffered by the Olson patent that the disappearance of said fish will cause disappointment with the infant and thus provide an opportunity for the caregiver to comment as to why the fish disappeared. In this way, the caregiver may discuss the toilet training process in context of the disappearing fish as opposed to the fault of the infant. While this approach may prove effective with some infants, not all infants may find the disappearance of non-anthropomorphic fish to be emotionally impactful enough to alter their behavior. So while the use of non-anthropomorphic objects (i.e., fish) helps to create a more realistic scene of interactively-interrelated graphics, it may not prove to be as effective in toilet training.

What is needed is a disposable absorbent article having wetness indicating graphics which include active anthropomorphic characters so as to provide a more impactful toilet training experience.

### SUMMARY OF THE INVENTION

An absorbent article having an outer cover which has an interior surface and an opposite exterior surface; an absorbent assembly disposed on the interior surface; a permanent character graphic disposed on the outer cover; a permanent object graphic disposed on the outer cover; wherein the permanent character graphic is interactively interrelated with the permanent object graphic; and further having at least one active character graphic, wherein said active character graphic is related in subject matter with said permanent character graphic and said permanent object graphic. The permanent character graphic may be illustrated to be involved in an activity utilizing the permanent object graphics, and the permanent object graphics are the subject of the activity. The active character graphic may become significantly more visible when exposed to urine. Alternatively, the active character graphic may become significantly more visible with the passage of time when exposed to the environment but not exposed to urine. Alternatively, the active character graphic may become significantly less visible when exposed to urine. Alternatively, the active character graphic may become significantly less visible with the passage of time when exposed to the environment but not exposed to urine.

The absorbent article may also have a feel-wet liner which provides a feel-wet indication to the wearer or caregiver. The active character graphic may further visually communicate the feel-wet indication. The active character graphic may change to a different color to denote a particular triggered response.

Alternatively, an absorbent article having an outer cover having an interior surface and an opposite exterior surface; an absorbent assembly disposed on the interior surface; a permanent graphic disposed on the outer cover; and at least one active character graphic, wherein said active character graphic is related in subject matter with said permanent graphic. The permanent character graphic may be a permanent character graphic or a permanent object graphic.

### BRIEF DESCRIPTION OF THE DRAWINGS

While the specification concludes with claims pointing out and distinctly claiming the present invention, it is believed the same will be better understood by the following drawings taken in conjunction with the accompanying specification wherein like components are given the same reference number.

FIG. 1 illustrates a front perspective view of a training pant incorporating the principles of the present invention, showing both permanent graphics and active graphics;

FIG. 2 illustrates a rear perspective view of the training pant of FIG. 1;

FIG. 3 illustrates a top plan, partially disassembled view of the training pant of FIGS. 1 and 2, in a stretched and laid flat condition and with portions broken away for purposes of illustration;

FIG. 4 illustrates a front perspective view similar to FIG. 1, although showing only the permanent graphics;

FIG. 5a illustrates a front perspective view of an alternative embodiment of a training pant incorporating the principles of the present invention;

FIG. 5b illustrates a rear perspective view of the training pant of FIG. 5a;

FIG. 6a illustrates a front perspective view of an alternative embodiment of a training pant incorporating the principles of the present invention;

FIG. 6b illustrates a rear perspective view of the training pant of FIG. 6a;

FIG. 7a illustrates a front perspective view of an alternative embodiment of a training pant incorporating the principles of the present invention;

FIG. 7b illustrates a rear perspective view of the training pant of FIG. 7a;

FIG. 8a illustrates a front perspective view of an alternative embodiment of a training pant incorporating the principles of the present invention;

FIG. 8b illustrates a rear perspective view of the training pant of FIG. 8a;

FIG. 9a illustrates a front perspective view of an alternative embodiment of a training pant incorporating the principles of the present invention;

FIG. 9b illustrates a rear perspective view of the training pant of FIG. 9a;

FIG. 10a illustrates a front perspective view of an alternative embodiment of a training pant incorporating the principles of the present invention;

FIG. 10b illustrates a rear perspective view of the training pant of FIG. 10a;

FIG. 11a illustrates a front perspective view of an alternative embodiment of a training pant incorporating the principles of the present invention;

FIG. 11b illustrates a rear perspective view of the training pant of FIG. 1a;

FIGS. 12a, 12b and 12c illustrate various partial section views of absorbent articles according to the present invention;

FIG. 13 illustrates a front perspective view of an alternative embodiment of a training pant incorporating the principles of the present invention;

FIG. 14a illustrates a front perspective view of an alternative embodiment of a training pant incorporating the principles of the present invention; and

FIG. 14b illustrates a front perspective view of an alternative embodiment of a training pant incorporating the principles of the present invention.

### DETAILED DESCRIPTION OF THE DRAWINGS

As used herein, the following terms have the following meanings:

“Active graphic” as used herein refers to an appearing graphic, a fading graphic, or a combination of appearing and fading graphics.

“Appearing graphic” is used herein to refer to a graphic that becomes visible or becomes significantly more visible when exposed to urine, or that becomes visible or becomes significantly more visible with the passage of time when exposed to the environment but not exposed to urine.

“Fading graphic” is used herein to refer to a graphic that becomes invisible or significantly less visible when exposed to urine, or that becomes invisible or significantly less visible with the passage of time when exposed to the environment but not exposed to urine.

“Permanent graphic” is used herein to refer to a graphic that does not substantially change its degree of visibility when the absorbent article is insulted with urine and when the absorbent article is exposed to the environment, in simulated use conditions. The change in visibility of a graphic or a portion of a graphic can be determined based on a person's observation of the graphic before and after the article containing the graphic is exposed to liquid. For purposes hereof, an article is exposed to liquid by immersing the article completely in an aqueous solution containing 0.9 weight percent sodium chloride, used at room temperature (approximately 23° C.), for a period of twenty minutes. After 20 minutes the product is removed from the aqueous solution and placed on a TEFLON™ coated fiberglass screen having 0.25 inch openings, which is commercially available from Taconic Plastics Inc., Petersberg, N.Y., USA, which in turn is placed on a vacuum box and covered with a flexible rubber dam material. A vacuum of 3.5 kilopascals (0.5 pounds per square inch) is drawn in the vacuum box for a period of 5 minutes, after which the article is removed and observed. The person with normal or corrected vision of 20-20 should make the observations from a distance of 1 meter in an environment providing 30 footcandles (320 Lux) of illumination. Changes in the visibility of the graphic should be identified, and distinguished where necessary from changes in the color of other materials such as fluff pulp within an absorbent assembly. Desirably, the permanent graphic can be configured so that the entire graphic also does not substantially change its appearance, size or shape when the product is insulted with liquid or exposed to the environment.

“Character graphic” is used herein to refer to a graphic containing an anthropomorphous image, and in particular an image having or suggesting human form or appearance which ascribes human motivations, characteristics or behavior to inanimate objects, animals, natural phenomena, cartoon characters, or the like.

“Object graphic” is used herein to refer to a graphic representing an object or thing, which can include an inanimate object or an alpha-numeric character (e.g., the letter “J”, the number “3”).

“Interactively interrelated” is used to mean that the character graphic is illustrated to be involved in or performing an action or activity, and the object graphic is the object of or is associated with the action or activity.

“Related in subject matter” refers to the situation where the subject matter of one graphic is the same as or is linked to the subject matter of another graphic. The subject matter relationship can be between two or more text messages, between two or more pictorial images, or between a combination of one or more text messages and one or more pictorial images. By way of example, two pictorial images are considered related in subject matter where the images are identical; separately illustrate different sizes, shapes, colors of a common object; each illustrate one and the other of two objects that are commonly associated with one another, such as the moon and stars, a body of water and water toys, a sandbox and suitable toys, a baseball bat and bail, a barn and animals, or the like; illustrate different items used in a particular activity, such as a sporting activity, a gardening activity or the like; jointly illustrate geometrically mating or engaging elements such as a triangle and a triangularly-shaped aperture, or two halves of a zipper; each illustrate one part of a multipart picture; or the like. Similarly, two text messages are considered related in subject matter where the messages: are identical; jointly form a sentence, thought, or action such as “jump” and “up”; each refer to one and the other of two items that are commonly associated with one another, such as “bat” and “ball,” “Big” and “Kid,” “Big” and “Girl,” or “Big” and “Boy”; jointly present a question and answer; or the like. Likewise, a text message and a pictorial image are considered to be related in subject matter where the text names, defines or describes the image; or the like.

“Text message” means a graphic consisting of one or more alphanumeric symbols.

“Pictorial image” means a graphic consisting of one or more pictures.

“Graphic” is used herein to mean any design, pattern, or the like that is or becomes visible on an absorbent article, and specifically includes text messages that consist of one or more alphanumeric symbols, pictorial images that consist of one or more pictures, and combinations thereof.

“Liquid communication” means that liquid such as urine is able to travel from one layer or element to another layer or element.

“Water-permeable” and “water-impermeable” are used herein to refer to the penetrability of materials in the context of the intended usage of disposable absorbent articles. Specifically, the term “water-permeable” refers to a layer or a layered structure having pores, openings, and/or interconnected void spaces that permit liquid water to pass through its thickness in the absence of a forcing pressure. Conversely, the term “water-impermeable” refers to a layer or a layered structure through the thickness of which liquid water cannot pass in the absence of a forcing pressure. A layer or a layered structure that is water-impermeable according to this definition may be permeable to water vapor, i.e., may be “vapor-permeable”. As is well known in the art, a common method for measuring the permeability to water of the materials typically used in absorbent articles is a hydrostatic pressure test, also called a hydrostatic head test or simply a “hydrohead” test. Suitable well known compendial methods for hydrohead testing are approved by INDA (formerly the International Nonwovens and Disposables Association, now The Association of the Nonwoven Fabrics Industry) and EDANA (European Disposables And Nonwovens Association).

“Absorbent article” refers to devices that absorb and contain liquid, and more specifically, refers to devices that are placed against or in proximity to the body of the wearer to absorb and contain the various exudates discharged from the body.

“Disposable” is used herein to describe absorbent articles that generally are not intended to be laundered or otherwise restored or reused as an absorbent article (i.e., they are intended to be discarded after a single use and, preferably, to be recycled, composted or otherwise disposed of in an environmentally compatible manner).

“Pant” used herein refers to disposable absorbent articles having a waist opening and leg openings designed for infant or adult wearers. A pant may be placed in position on the wearer by inserting the wearer's legs into the leg openings and sliding the pant into position about the wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the term “pant” is used herein, pants are also commonly referred to as “closed diapers”, “prefastened diapers”, “pull-on diapers”, “training pants” and “diaper-pants”. Suitable pants are disclosed in U.S. Pat. No. 5,246,433, issued to Hasse, et al. on Sep. 21, 1993; U.S. Pat. No. 5,569,234, issued to Buell et al. on Oct. 29, 1996; U.S. Pat. No. 6,120,487, issued to Ashton on Sep. 19, 2000; U.S. Pat. No. 6,120,489, issued to Johnson et al. on Sep. 19, 2000; U.S. Pat. No. 4,940,464, issued to Van Gompel et al. on Jul. 10, 1990; U.S. Pat. No. 5,092,861, issued to Nomura et al. on Mar. 3, 1992; U.S. patent application Ser. No. 10/171,249, entitled “Highly Flexible And Low Deformation Fastening Device”, filed on Jun. 13, 2002; U.S. Pat. No. 5,897,545, issued to Kline et al. on Apr. 27, 1999; U.S. Pat. No. 5,957,908, issued to Kline et al on Sep. 28, 1999, the disclosure of each of which is incorporated herein by reference.

“Training pant” is a pant which provides toilet training utility.

“Longitudinal” is a direction running parallel to the maximum linear dimension of the article and includes directions within ±45° of the longitudinal direction.

“Lateral” or “transverse” direction is orthogonal to the longitudinal direction.

“Z-direction” is orthogonal to both the longitudinal and transverse directions.

“X-Y plane” refers to the plane congruent with the longitudinal and transverse directions.

“Disposed” is used to mean that an element(s) is formed (joined and positioned) in a particular place or position as a unitary structure with other elements or as a separate element joined to another element.

“Joined” encompasses configurations whereby an element is directly secured to another element by affixing the element directly to the other element, and configurations whereby an element is indirectly secured to another element by affixing the element to intermediate member(s) which in turn are affixed to the other element.

“Unitary” absorbent article refers to absorbent articles which are formed of separate parts united together to form a coordinated entity so that they do not require separate manipulative parts like a separate holder and liner.

“Diaper” refers to an absorbent article generally worn by infants and incontinent persons about the lower torso.

“Proximal” and “distal” refer respectively to the location of an element near to or far from the center of a structure, e.g., the proximal edge of a longitudinally extending element is located nearer to the longitudinal axis than the distal edge of the same element is located relative to the same longitudinal axis.

The principles of the present invention can be incorporated into a variety of absorbent articles, such as diapers, training pants, incontinence products, diaper pants, disposable underwear, or the like. The present invention is particularly suited for use with training pants or diaper pants to aid in toilet training. For ease of explanation, the description hereafter will be in terms of a child's training pant.

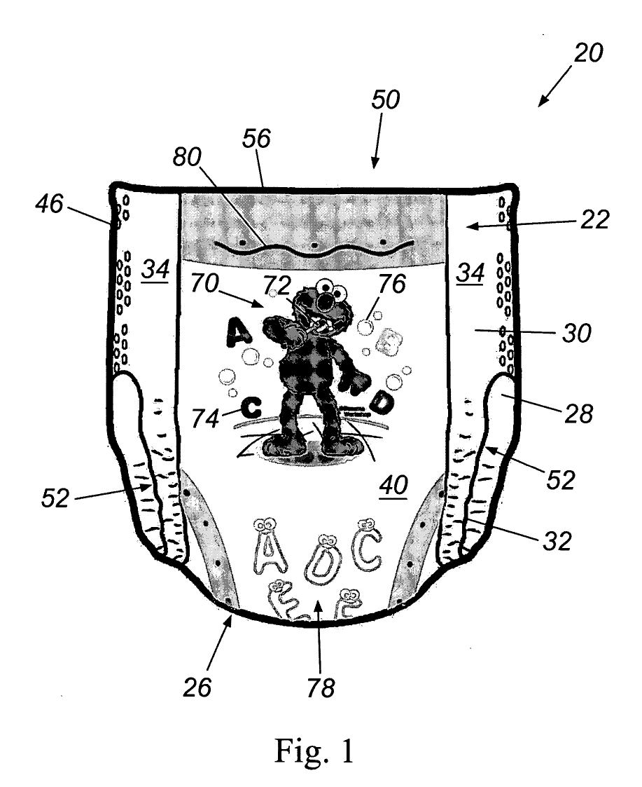

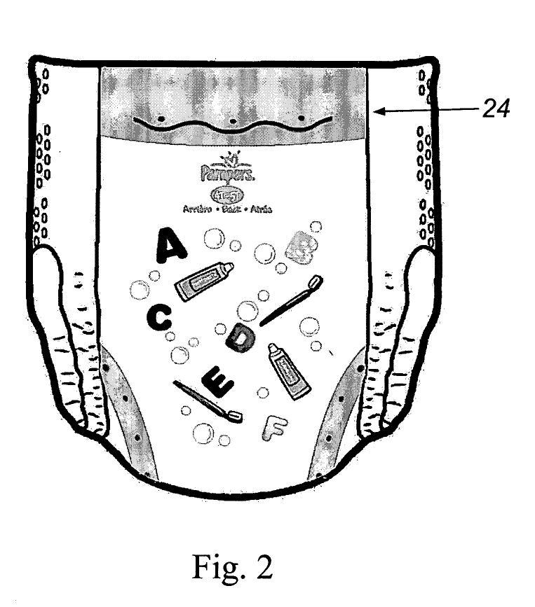

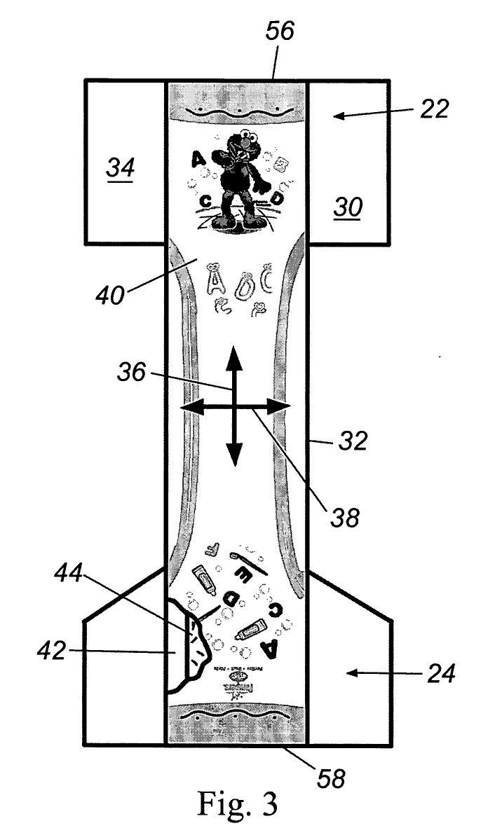

A training pant 20 is illustrated in a fully assembled condition in FIGS. 1 and 2 and in a partially disassembled, stretched and laid flat condition in FIG. 3. The training pant 20 defines a first or front waist region 22, a second or back waist region 24, a crotch region 26 positioned between and interconnecting the front and back waist regions, an inner surface 28 which is configured to contact the wearer, and an outer surface 30 opposite the inner surface which is configured to contact the wearer's clothing. The illustrated training pant 20 comprises an absorbent chassis 32 and a plurality of transversely opposed side panels 34. The absorbent chassis 32 and side panels 34 can be integrally formed or comprise two or more separate elements, as shown.

Referring now to FIG. 3, the training pant 20 defines a longitudinal centerline 36, a transverse centerline 38, a first or front longitudinal end edge 56, and a second or back longitudinal end edge 58. The first waist region 22 abuts the first longitudinal end edge 56, and the second waist region 24 abuts the second longitudinal end edge 58. The article as illustrated is longer in the longitudinal direction than in the transverse direction. The illustrated absorbent chassis 32 comprises an outer cover 40 and a bodyside liner 42 which is connected to the outer cover in a superposed relation. The absorbent chassis 32 also comprises an absorbent assembly 44 which is located between the outer cover and the bodyside liner. The absorbent chassis 32 is configured to contain and/or absorb any body exudates discharged from the wearer. Additionally, a pair of containment flaps (not shown) may be joined to said absorbent chassis.

With the training pant 20 in a fully assembled condition as illustrated in FIGS. 1 and 2, the front and back waist regions 22 and 24 are joined together by side seams 46 to define a waist opening 50 and a pair of leg openings 52. The front waist region 22 comprises the portion of the training pant 20 which, when worn, is positioned on the front of the wearer while the back waist region 24 comprises the portion of the training pant which, when worn, is positioned on the back of the wearer. The crotch region 26 of the training pant 20 comprises the portion of the training pant which, when worn, is positioned between the legs of the wearer and covers the lower torso of the wearer. The side panels 34 comprise the portions of the training pant 20 which, when worn, are positioned on the side hip regions of the wearer. The longitudinal end edges 56 and 58 of the training pant 20 are configured to encircle the waist of the wearer when worn and provide the waist opening 50.

The outer cover 40 has an exterior surface corresponding to the outer surface 30 of the training pant and an opposite interior surface. The outer cover 40 desirably comprises a material that is substantially liquid impermeable. The outer cover 40 can be a single layer of liquid impermeable material, but desirably comprises a multi-layered laminate structure in which at least one of the layers is liquid impermeable. For instance, the outer cover 40 can include a liquid permeable outer layer and a liquid impermeable inner layer that are suitably joined together by a laminate adhesive (not shown). Suitable laminate adhesives, which can be applied continuously or intermittently as beads, a spray, parallel swirls, or the like, can be obtained from Findley Adhesives, Inc., of Wauwatosa, Wis., or from National Starch and Chemical Company, Bridgewater, N.J. The liquid permeable outer layer can be any suitable material and desirably one that provides a generally cloth-like texture. One example of such a material is a 20 gsm (grams per square meter) spunbond polypropylene nonwoven web. The outer layer can also be made of those materials of which liquid permeable bodyside liner 42 is made. While it is not a necessity for outer layer to be liquid permeable, it is desired that it provides a relatively cloth-like texture to the wearer.

The inner layer of the outer cover 40 can be both liquid and vapor impermeable, or can be liquid impermeable and vapor permeable. The inner layer is desirably manufactured from a thin plastic film although other flexible liquid impermeable materials can also be used. The inner layer, or the liquid impermeable outer cover 40 when a single layer, prevents waste material from wetting articles, such as bedsheets and clothing, as well as the wearer and caregiver. If the outer cover 40 is a single layer of material, it can be embossed and/or matte finished to provide a more cloth-like appearance. As earlier mentioned, the liquid impermeable material can permit vapors to escape from the interior of the disposable absorbent article, while still preventing liquids from passing through the outer cover 40. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn.

Another suitable liquid impermeable film for use as liquid impermeable inner layer, or a single layer liquid impermeable outer cover 40, is a 0.025 millimeter (1.0 mil) polyethylene film commercially available from Edison Plastics Company of South Plainfield, N.J. In preferred embodiments, the liquid impermeable film comprises a laminate of a nonwoven and a thin plastic film such as a thermoplastic film having a thickness of about 0.012 mm (0.5 mil) to about 0.051 mm (2.0 mils). Suitable liquid impermeable films include those manufactured by Tredegar Industries Inc. of Terre Haute, Ind. and sold under the trade names X15306, X10962, and X10964. Other suitable liquid impermeable films may include breathable materials that permit vapors to escape from the liquid impermeable film while still preventing exudates from passing through the liquid impermeable film. Exemplary breathable materials may include materials such as woven webs, nonwoven webs, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Application No. WO 95/16746, published on Jun. 22, 1995 in the name of E. I. DuPont and copending U.S. patent application Ser. No. 08/744,487, filed on Nov. 6, 1996 in the name of Curro. Other breathable liquid impermeable films including nonwoven webs and apertured formed films are described in U.S. Pat. No. 5,571,096 issued to Dobrin et al. on Nov. 5, 1996. An exemplary, suitable liquid impermeable film is disclosed in U.S. Pat. No. 6,107,537 entitled “Disposable absorbent articles providing a skin condition benefit” issued to Elder et al on Aug. 22, 2000. Other suitable materials and/or manufacturing techniques may be used to provide a suitable liquid impermeable film including, but not limited to, surface treatments, particular film selections and processing, particular filament selections and processing, etc. Each of these references is hereby incorporated by reference herein.

As shown in FIGS. 1-3, the training pant 20, and in particular the outer cover 40, desirably comprises one or more appearance-related components. Examples of appearance-related components include, but are not limited to, graphics highlighting or emphasizing leg and waist openings in order to make product shaping more evident or visible to the user; graphics highlighting or emphasizing areas of the product to simulate functional components such as elastic leg bands, elastic waistbands; graphics highlighting areas of the product to change the appearance of the size of the product; registering wetness indicators, temperature indicators, and the like in the product; registering a back label, or a front label, in the product; and registering written instructions at a desired location in the product.

The illustrated training pant 20 includes registered outer cover graphics, including interactive wetness indicating graphics. More specifically, the training pant includes a permanent character graphic 70 in the form of Elmo™ having human-like expressions and brushing his teeth; permanent object graphics 72, 74 and 76 in the form of a toothbrush, alphabetic letters (e.g., the letter “C”), and bubbles, respectively; and a plurality of active object graphics 78 in the form of alphabet-friends. The outer cover graphics also include a simulated elastic waistband 80 and simulated elastic leg bands 84, both of which can be permanent graphics.

The active character graphics 78 can be disposed on the interior surface of the outer cover 40 and are visible from the exterior surface of the outer cover. The active character graphics 78 are in liquid communication with the absorbent assembly 44, meaning that liquid such as urine is capable of moving between the active character graphic and the absorbent assembly under ordinary use conditions.

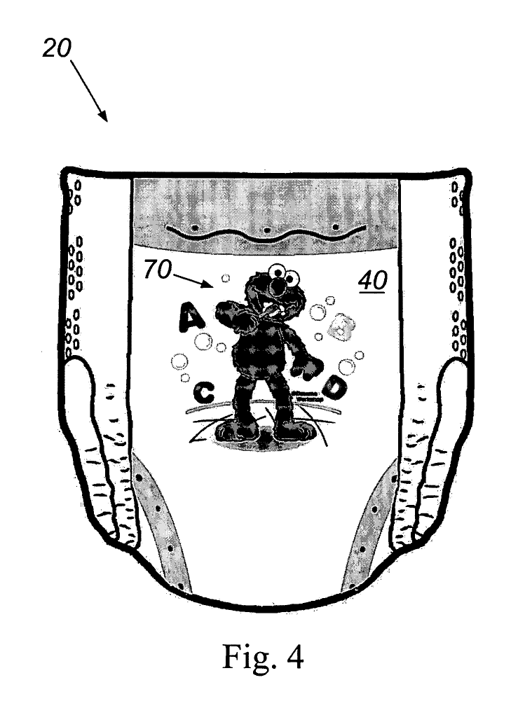

When the child wets the training pant 20, liquid is communicated to the active character graphics 78, whereupon the character graphics either dissolve, change color, appear, or the like. The effect is illustrated in FIG. 4, showing the situation where the active character graphics 78 disappear upon contact with urine. Where appearing graphics are employed, the situation would work in reverse and the appearance of the pant would change from FIG. 4 to FIG. 1 upon liquid insult. Alternatively, the active character graphics 78 can comprise appearing graphics that are triggered upon use by exposure to the environment.

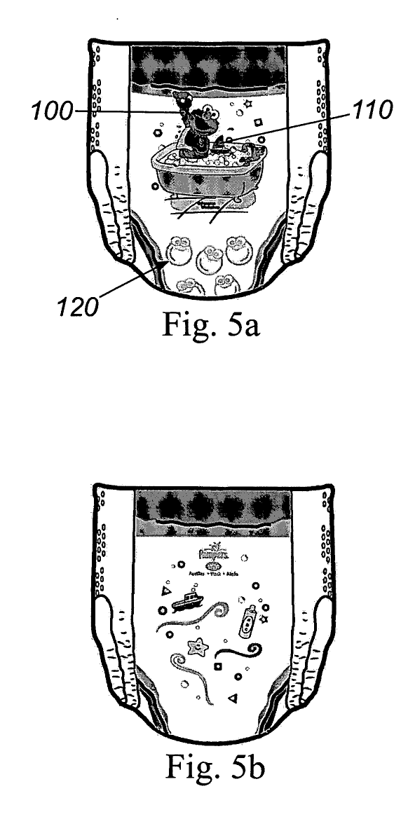

FIGS. 5-11 depict additional exemplary, non-limiting embodiments of the present invention. More specifically, FIG. 5a shows Elmo™ (permanent character graphic 100) taking a bath with a sailboat (permanent object graphic 110) and subject-matter-related bubble-friends (active character graphics 120). Herein, a caregiver may say “Elmo is making taking a bath with his bubble-friends, make sure you tell me when you need to go potty so that you don't make his bubble-friends disappear or go away.” FIG. 5b illustrates an exemplary, rear perspective view of the training pant of FIG. 5a.

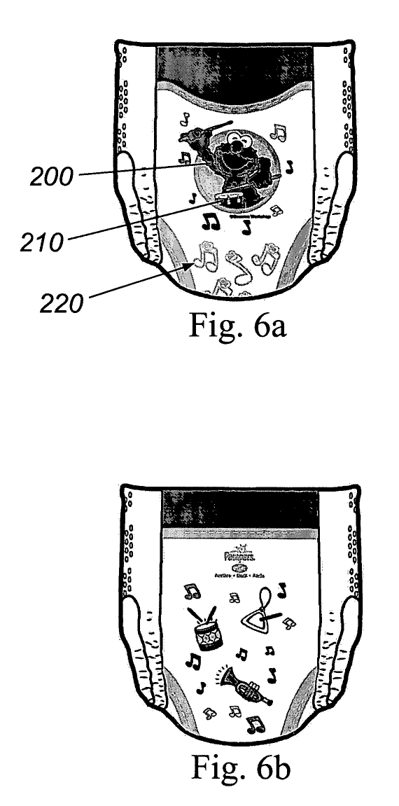

FIG. 6a shows Elmo™ (permanent character graphic 200) playing the drum (permanent object graphic 210) and subject-matter-related musical-note-friends (active character graphics 220). Herein, a caregiver may say “Elmo is making music with his musical-note-friends, make sure you tell me when you need to go potty so that you don't make his musical-note-friends disappear or go away.” FIG. 6b illustrates an exemplary, rear perspective view of the training pant of FIG. 6a.

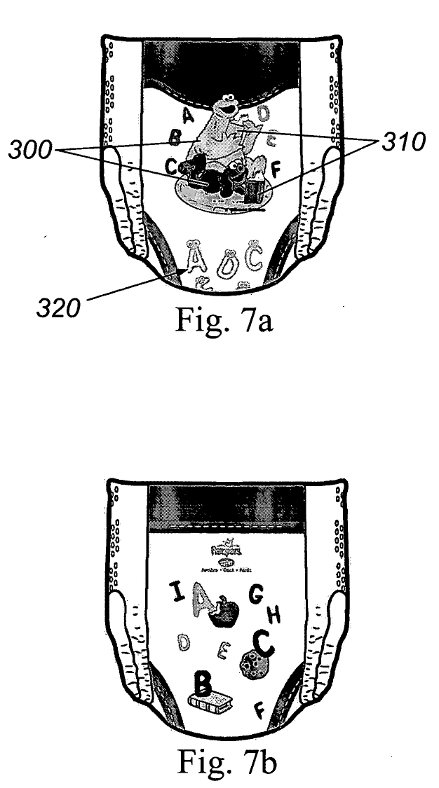

FIG. 7a shows Elmo™ and Cookie Monster™ (permanent character graphics 300) reading a book (permanent object graphic 310) and subject-matter-related alphabet-friends (active character graphic 320). Herein, a caregiver may say “Elmo and Cookie are having fun reading with their alphabet-friends, make sure you tell me when you need to go potty so that you don't make their alphabet-friends disappear or go away.” FIG. 7b illustrates an exemplary, rear perspective view of the training pant of FIG. 7a.

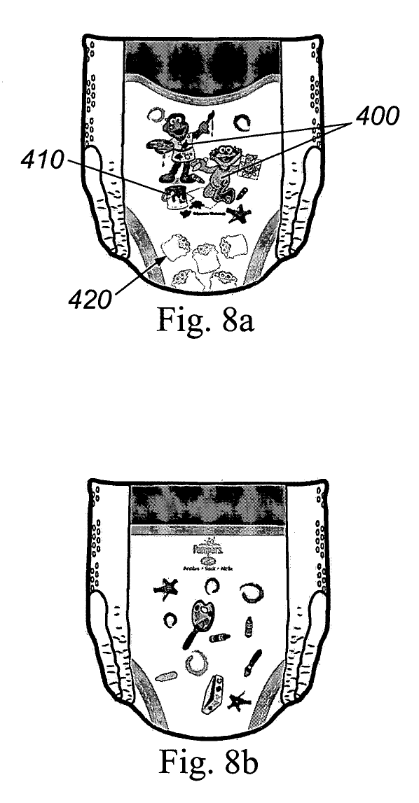

FIG. 8a shows Elmo™ and Zoe™ (permanent character graphics 400) painting from paint buckets (permanent object graphics 410) and subject-matter-related paint-bucket-friends (active character graphics 420). Herein, a caregiver may say “Elmo, Zoe, and the paint-bucket-friends are painting lots of fun pictures, make sure you tell me when you need to go potty so that you don't make their paint-bucket-friends disappear or go away.” FIG. 8b illustrates an exemplary, rear perspective view of the training pant of FIG. 8a.

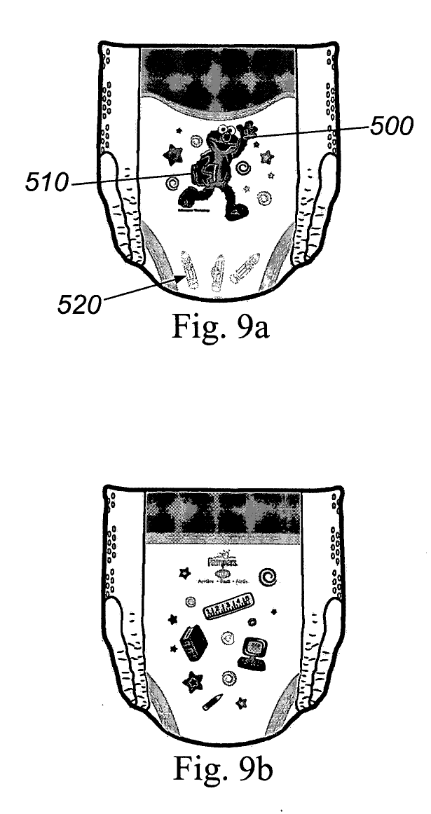

FIG. 9a shows Elmo™ (permanent character graphic 500) carrying his school backpack (permanent object graphic 510) and subject-matter-related pencil-friends (active character graphics 520). Herein, a caregiver may say “Elmo and his pencil-friends are walking to school, stay dry so you don't make Elmo's pencil-friends disappear.” FIG. 9b illustrates an exemplary, rear perspective view of the training pant of FIG. 9a.

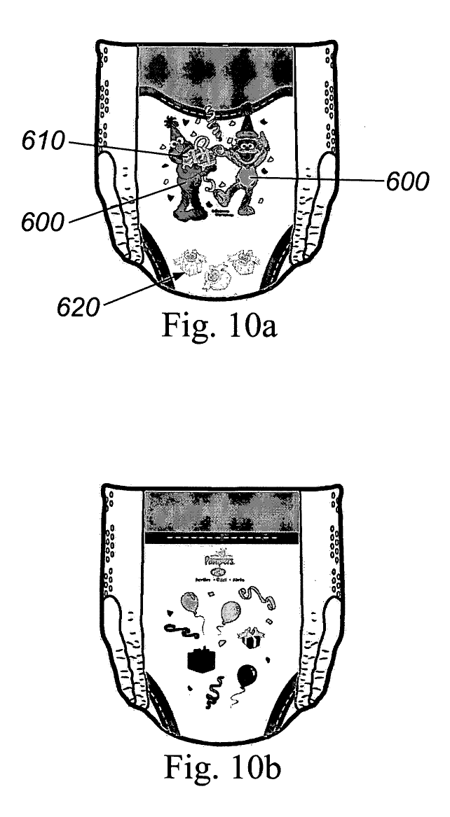

FIG. 10a shows Elmo™ and Zoe™ (permanent character graphics 600) opening a birthday gift (permanent object graphic 610) and subject-matter-related gift-friends (active character graphics 620). Herein, a caregiver may say “Make sure you stay dry so Elmo and Zoe can have fun at the party with their gift-friends.” FIG. 10b illustrates an exemplary, rear perspective view of the training pant of FIG. 10a.

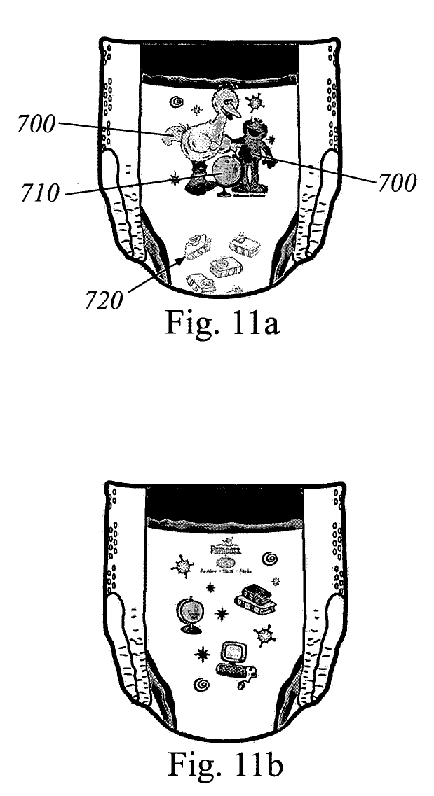

FIG. 11a shows Elmo™ and Big Bird™ (permanent character graphics 700) reading a globe (permanent object graphic 710) and subject-matter-related geography-book-friends (active character graphics 720). Herein, a caregiver may say “Make sure you stay dry so Elmo and Big Bird can learn about the world with their geography-book-friends.” FIG. 11b illustrates an exemplary, rear perspective view of the training pant of FIG. 11a.

Considerations for General Construction of the Subject Training Pant:

A. Graphics Generally:

The graphics of the absorbent article can be constructed to provide a story line involving a permanent character graphic and a permanent object graphic along with an active character graphic which is related in subject matter with said storyline.

Ideally, the permanent character graphic would be suitable for children's underwear and could be utilized to motivate children to wear the pants and use a potty or toilet. To that end, the permanent character graphic can be associated with popular characters in the media, advertising or well known in a particular culture. Ideally, they are characters that the child or caregiver care about and want to identify with. Ideally the child can imagine himself or herself taking the place of the permanent character graphic.

The active character graphic may prove helpful in motivating a child to become toilet trained. The active character graphic can also give parents and caregivers an interactive training element for use during toilet training. The use of an active character graphic facilitates non-threatening and gentle communications between the child and caregiver when the child has had an accident and wet his or her pants. The active character graphic is a third party that the caregiver can use to deflect blame and shame from the child while still communicating the desire that the child not wet his or her pants. It may be easier for the child to accept criticism when the caregiver seems to be talking about a third person. It may also be less stressful for the caregiver because he or she does not need to directly confront the child over the mistake.

In some conventional approaches, the disappearance of non-anthropomorphic fish are used to serve as active object graphics to create a more realistic scene of interactively-interrelated graphics; however, it is believed that the use of active character graphics (i.e., anthropomorphic graphics) would prove more effective in toilet training. Without wishing to be bound by theory, it is believed that the anthropomorphic objects gain more initial attention from the children because they are not truly indigenous to the scene (e.g., bath bubbles with a face); in a sense they are Muppet®-ized. As such, their disappearance (or appearance) is more impactful; hence, more effective in toilet training.

B. Inks

Suitable urine-soluble inks are available from a variety of commercial vendors, such as Sun Chemical Corp. of Philadelphia, Pa., USA under the trade designation AQUA DESTRUCT.

Particular urine-soluble compositions are disclosed in U.S. Pat. No. 4,022,211 issued May 10, 1977 to Timmons et al., which is incorporated herein by reference. The ink color can be selected to provide a pleasing appearance and graphic impact, including fading rapidly upon contact with liquid. To facilitate rapid fading, the fading graphics can comprise line drawings having a line width of from about 1 to about 2 millimeters.

The active graphic can also comprise a fading or an appearing graphic which is formed from a composition such as an ink or adhesive that changes color when exposed to an aqueous solution such as urine. A color change composition can be adapted to blend in with a background or surrounding color, either before or after exposure to the aqueous solution. Suitable compositions of this color-change type are available from a variety of commercial vendors, such as a pH-change/color-change hot melt adhesive available from Findley Adhesives, Inc. of Wauwatosa, Wis., USA. Alternatively, the active graphic can comprise pH sensitive inks, fugitive inks, colored absorbent particles, hydratable salts, moisture sensitive films, enzymes, heat sensitive inks and dyes, or the like. Fading graphics can simply disappear from view, relative to the exterior surface of the outer cover. For example, fading graphics can be made to disappear into a permanent background graphic. The active graphic can also be configured to appear over time due to exposure to the environment. In particular, the active graphic can be responsive to time intervals, temperature levels, oxygen levels, or the like, and combinations thereof. Various visual indicators that appear over time in response to particular conditions are disclosed in U.S. Pat. No. 5,058,088 issued Oct. 15, 1991 to Haas et al.; U.S. Pat. No. 5,053,339 issued Oct. 1, 1991 to Patel; U.S. Pat. No. 5,045,283 issued Sep. 3, 1991 to Patel; U.S. Pat. No. 4,987,849 issued Jan. 29, 1991 to Sherman; U.S. Pat. No. 4,903,254 issued Feb. 20, 1990 to Haas; U.S. Pat. No. 4,812,053 issued Mar. 14, 1989 to Bhattacharjee; and U.S. Pat. No. 4,292,916 issued Oct. 6, 1981 to Bradley et al.; all of which are incorporated herein by reference. An active graphic that appears over time may be applied to the product when use is initiated, or formed as an integral component of the product.

In use, the active graphic can appear or fade when an accident occurs and urine comes into contact with the active graphic. Desirably, the active graphic appears or fades in about 3 minutes or less, particularly in about 1 minute or less, and more desirably in about 20 seconds or less, when the absorbent article is insulted with 200 milliliters or more of urine, and more desirably about 40 to about 60 milliliters or more of urine.

When the active graphic is a fading graphic, the training opportunity is based on the fact that the related subject matter has disappeared and thus altered the story line. A slight sense of disappointment may be gently communicated to the child, because the active character graphics are no longer present and the story line has changed. This design approach can be used as a motivational basis for teaching the child that it is within the child's control to permit the story line to continue unaltered for as long as the child can go without wetting his or her pants.

When the active graphic is an appearing graphic as opposed to a fading graphic, the training opportunity is based on the fact that the caregiver can explain the appearance of a new graphic for a reward, therefore a more positive approach can be taken. For example, the active graphic can be adapted to appear over time, and it may be viewed as a reward for the child for staying dry. It can in this case be used as a tool to identify toilet training queues. For instance, if an active graphic appeared over time, such as one every hour while staying dry, the caregiver could say “Hey, look how well you are doing; you stayed dry for 2 hours.” The active graphic can alternatively change color to a brighter and/or darker color. By way of illustration, a character graphic outlined and colored very pale, such as yellow or tan, can when in contact with urine turn into bright reds, greens, and/or blues. Alternatively, only the outline of the active graphic can be present and a pattern or solid color can appear over time or when in contact with urine. For example, the active graphic can include an outline of a party friend, which fills in to become a solid color when urine comes in contact with a party friend.

C. Absorbent Assembly:

The liquid permeable bodyside liner 42 generally overlies the outer cover 40 and absorbent assembly 44, and can but need not have the same dimensions as the outer cover 40. The bodyside liner 42 is desirably compliant, soft feeling, and non-irritating to the child's skin. Further, the bodyside liner 42 can be less hydrophilic than the absorbent assembly 44, to present a relatively dry surface to the wearer and permit liquid to readily penetrate through its thickness.

The bodyside liner 42 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics can be used for the bodyside liner 42. For example, the bodyside liner can be composed of a meltblown or spunbonded web of polyolefin fibers. The bodyside liner can also be a bonded-carded web composed of natural and/or synthetic fibers. The bodyside liner can be composed of a substantially hydrophobic material, and the hydrophobic material may, optionally, be treated with a surfactant or otherwise processed to impart a desired level of wettability and hydrophilicity. For example, the material can be surface treated with about 0.28 weight percent of a surfactant commercially available from the Rohm and Haas Co. under the trade designation Triton X-102. The surfactant can be applied by any conventional means, such as spraying, printing, brush coating or the like. The surfactant can be applied to the entire bodyside liner 42 or can be selectively applied to particular sections of the bodyside liner, such as the medial section along the longitudinal centerline. Other such hydrophobic treatments include, but are not limited to, the application of hydrophobic surface coating (as exampled in co-pending U.S. Patent Application Ser. No. 60/543,785, entitled “Hydrophobic Surface Coated Absorbent Articles And Associated Methods”, filed on Feb. 11, 2004) and flouro-treatment (as exampled in co-pending U.S. patent application Ser. No. 10/703,239, entitled “Disposable Absorbent Articles With Masking Topsheet”, filed on Nov. 7, 2003).

A suitable liquid permeable bodyside liner 42 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber available from CHISSO Corporation, Osaka, Japan. In this particular bicomponent fiber, the polypropylene forms the core and the polyethylene forms the sheath of the fiber. Other fiber orientations are possible, such as multi-lobe, side-by-side, end-to-end, or the like.

The absorbent assembly 44 (FIG. 3) is positioned between the outer cover 40 and the bodyside liner 42, which components can be joined together by any suitable means such as adhesives as is well known in the art. The absorbent assembly 44 can be any structure which is generally compressible, conformable, non-irritating to the child's skin, and capable of absorbing and retaining liquids and certain body wastes. The absorbent assembly 44 can be manufactured in a wide variety of sizes and shapes, and from a wide variety of liquid absorbent materials commonly used in the art.

In some embodiments, the absorbent assembly (i.e., core) may comprise a wide variety of liquid-absorbent materials commonly used in disposable diapers and other absorbent articles such as comminuted wood pulp, which is generally referred to as air felt. Examples of other suitable absorbent materials include creped cellulose wadding; melt blown polymers, including co-form; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials. Exemplary absorbent structures for use as the absorbent assemblies are described in U.S. Pat. No. 4,610,678 entitled “High-Density Absorbent Structures” issued to Weisman et al. on Sep. 9, 1986; U.S. Pat. No. 4,673,402 entitled “Absorbent Articles With Dual-Layered Cores” issued to Weisman et al. on Jun. 16, 1987; U.S. Pat. No. 4,834,735, entitled “High Density Absorbent Members Having Lower Density and Lower Basis Weight Acquisition Zones”, issued to Alemany et al. on May 30, 1989; U.S. Pat. No. 4,888,231 entitled “Absorbent Core Having A Dusting Layer” issued to Angstadt on Dec. 19, 1989; U.S. Pat. No. 5,137,537 entitled “Absorbent Structure Containing Individualized, Polycarboxylic Acid Crosslinked Wood Pulp Cellulose Fibers” which issued to Herron et al. on Aug. 11, 1992; U.S. Pat. No. 5,147,345 entitled “High Efficiency Absorbent Articles For Incontinence Management” issued to Young et al. on Sep. 15, 1992; U.S. Pat. No. 5,342,338 entitled “Disposable Absorbent Article For Low-Viscosity Fecal Material” issued to Roe on Aug. 30, 1994; U.S. Pat. No. 5,260,345 entitled “Absorbent Foam Materials For Aqueous Body Fluids and Absorbent Articles Containing Such Materials” issued to DesMarais et al. on Nov. 9, 1993; U.S. Pat. No. 5,387,207 entitled “Thin-Until-Wet Absorbent Foam Materials For Aqueous Body Fluids And Process For Making Same” issued to Dyer et al. on Feb. 7, 1995; U.S. Pat. No. 5,397,316 entitled “Slitted Absorbent Members For Aqueous Body Fluids Formed Of Expandable Absorbent Materials” issued to LaVon et al. on Mar. 14, 1995; and U.S. Pat. No. 5,625,222 entitled “Absorbent Foam Materials For Aqueous Fluids Made From high Internal Phase Emulsions Having Very High Water-To-Oil Ratios” issued to DesMarais et al. on Jul. 22, 1997.

D. Location and Size of Graphics:

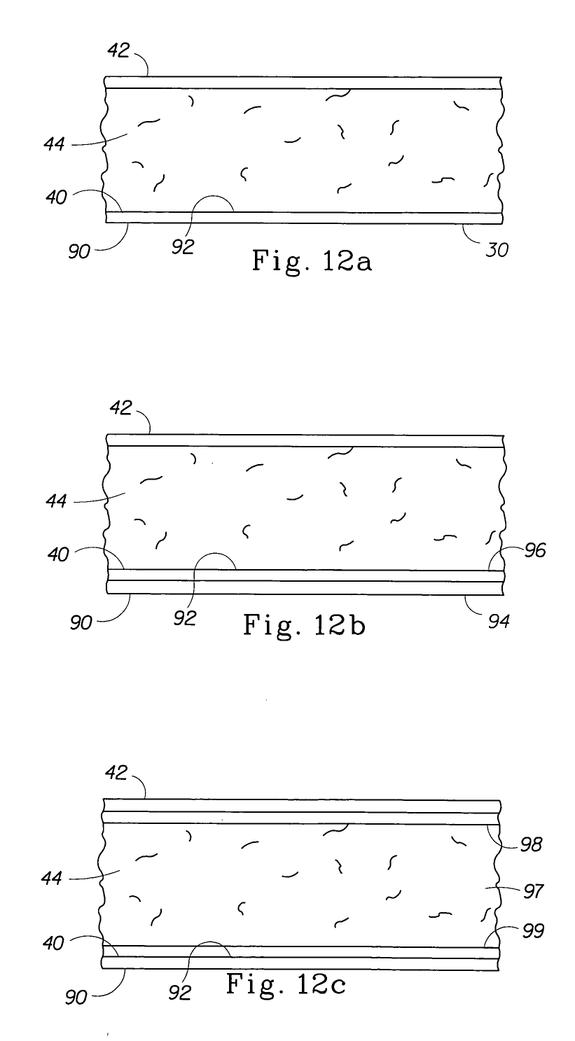

Various placements of the permanent and active graphics may be better understood with reference to the partial section views of absorbent articles that are shown in FIGS. 12A, 12B and 12C. The various layers of the illustrated embodiments can be secured together using adhesives, thermal bonds, mechanical bonds, or other means known to those skilled in the art.

FIG. 12a illustrates a partial section view of an absorbent article having an absorbent assembly 44 sandwiched between an outer cover 40 and a bodyside liner 42. The illustrated outer cover 40 consists of a single layer having an exterior surface 90 and an opposite interior surface 92. The permanent and active graphics can be disposed on the outer cover 40, which includes on either surface 90 or 92 of the outer cover, on the surface of the absorbent assembly 44 that faces the outer cover, or between the absorbent assembly and the outer cover. The outer cover 40 is desirably formed of a material that is liquid impermeable. In such embodiments and where the active graphics are triggered by contact with urine, it is desirable for the active graphics to be disposed on the interior surface 92 of the outer cover 40, on the surface of the absorbent assembly 44 that faces the outer cover, or between the absorbent assembly and the outer cover. The permanent and active graphics need not be located in the same position or on the same substrate.

FIG. 12b illustrates a partial section view of another absorbent article having an absorbent assembly 44 sandwiched between an outer cover 40 and a bodyside liner 42. The illustrated outer cover 40 consists of a two-layer composite comprising an outer layer 94 and an inner layer 96. The outer cover 40 has an exterior surface 90 and an opposite interior surface 92. The permanent and active graphics can be disposed on the outer cover 40, which includes, in particular, on the exterior surface 90, on the interior surface 92, between the outer and inner layers 94 and 96, on either or both facing surfaces of the outer and inner layers 94 and 96, on the surface of the absorbent assembly 44 that faces the outer cover, or between the absorbent assembly and the outer cover.

FIG. 12c illustrates a partial section view of a further absorbent article having an absorbent assembly 44 sandwiched between an outer cover 40 and a bodyside liner 42. The illustrated absorbent assembly includes a liquid storage layer 97 sandwiched between absorbent assembly substrates 98 and 99. The substrates 98 and 99 can comprise tissue wrap sheets, liquid distribution or liquid handling layers, or the like. The illustrated outer cover 40 consists of a single layer having an exterior surface 90 and an opposite interior surface 92, but can alternatively comprise a multiple-layer composite. The permanent and active graphics can be disposed on the outer cover 40, which includes specific locations such as on the exterior surface 90, on the interior surface 92, on either surface of the substrate 99 that is placed with or placed near the outer cover, between the substrate 99 and the outer cover, on the surface of the liquid storage layer 97 that faces the outer cover, or between the liquid storage layer and the substrate 99. Various other locations for the permanent and active graphics can be suitable where different configurations of the absorbent assembly 44 and outer cover 40 are employed, including but not limited to within a layer of the absorbent assembly or outer cover.

A permanent character graphic can be positioned in the front waist region and the back waist region along or near the longitudinal centerline of the product. In particular embodiments, a front view of the permanent character graphic is shown in the front waist region and a back view of the same permanent character graphic is shown in the back waist region. The permanent character graphic can be spaced from the first and second end edges of the product, for example, approximately 5 centimeters from the front end edge.

To provide proper focus on the active character graphic, the active character graphic can be positioned in the front crotch region along or near the longitudinal centerline of the product. In one particular embodiment, all of the active character graphics are located in a distinct active graphic region where the child can focus attention. The active graphic region suitably has a length dimension measured parallel to the longitudinal centerline of about 10 centimeters, more particularly about 9 centimeters, and a width dimension measured parallel to the transverse centerline of about 6 centimeters. The active graphic region may, for instance, be spaced from the first end edge of the product by approximately 13 to 16 centimeters.

There can be one or more active character graphics. It is believed desirable to use a plurality of active character graphics in order provide additional impact to the child. In particular embodiments, a training pant comprises 3 or more active character graphics, particularly from 4 to 8, such as 5 arranged in a 2-1-2 pattern.

The size of the active character graphics may depend in part on their number and type. It is generally preferred that the active character graphics have a size of at least about 1.5 by 1.5 centimeter and less than about 3 by 3 centimeters, and particularly at least about 1.8 by 1.8 centimeter and less than about 2.5 by 2.5 centimeters.

The overall size of the permanent character graphic can be significantly larger than the overall size of each discrete active object graphic. In particular embodiments, for instance, the permanent character graphic has a size measured by overall surface area that is at least twice as large, and more particularly at least three times as large, as the size of a single active character graphic. The maximum length dimension of the permanent character graphic, measured parallel to the longitudinal centerline, is desirably about 8 to about 9 centimeters.

The active character graphics, the permanent character graphics and the permanent object graphics are suitably disposed on the outer cover. Thus, the graphics can be formed or applied directly or indirectly to a surface of the outer cover, formed or applied between layers of a multiple layer outer cover, formed or applied to a substrate that is placed with or near the outer cover, formed or applied within a layer of the outer cover or another substrate, or other variations or combinations thereof. In particular embodiments, the graphics can be printed, sprayed, or otherwise applied directly on a layer of the outer cover. In other embodiments, the graphics can be applied to a layer placed with or near the outer cover, such as a substrate associated with the absorbent assembly, including but not limited to tissue layers, liquid handling layers, absorbent layers, or the like.

The permanent graphics can be located on the exterior surface of the outer cover to enhance the visual impact of the permanent graphics. Alternatively, however, the permanent graphics can be located on the interior surface of the outer cover or between layers of a multilayer outer cover, provided the permanent graphics remain visible from the exterior of the product.

The active graphics can be located on the interior surface of the outer cover, which is particularly desirable for active graphics that are triggered by urine in order to enhance the speed at which the graphics are contacted with urine and thus change their visual appearance. The outer cover desirably comprises a material that is formed or treated to be liquid impermeable. In alternative embodiments, the outer cover can comprise a material that is formed or treated to be at least partially liquid permeable. In this latter case, the active graphics can be located between layers of a multilayer outer cover, or, particularly for appearing graphics, on the exterior surface of the outer cover. Regardless of location, fading graphics should be visible from the exterior of the product before activation and appearing graphics should be visible from the exterior of the product after activation.

The outer cover can comprise a translucent material that has sufficient opacity, or a transparent or translucent material that is otherwise treated, to mask a urine soluble ink after it has dissolved. Such an outer cover should not be so opaque that the graphics printed on the interior surface of the outer cover or adjacent to the interior surface are obscured. Polymer films used to form the outer cover can be treated with titanium dioxide to make the film appear white and to develop sufficient opacity to mask urine, BM and dissolved inks. Examples of suitable outer cover materials include films formed of polyethylene, polypropylene, cataloy, bi-component, any polymer based extruded film, or the like. One such film is a polyethylene film having a thickness of about 0.2 millimeter (0.75 mil).

The permanent and active graphics can be formed on or applied to the outer cover or another substrate bonded to or placed with or placed near the outer cover by any suitable technique. The graphics are desirably registered with other components of the absorbent article during manufacture such that the graphics are positioned in the desired regions of the product.

For active graphics that are triggered by contact with urine, the active graphic can be in liquid communication with the absorbent assembly of the product. The absorbent assembly can but need not include a slot or densified region, incorporate a liquid distribution layer, or the like, to channel or direct liquid to the location near the outer cover where the active graphics are located.

As noted previously, liquid soluble inks can be used to form the active graphics. It is theorized that migration of the dissolved inks away from the outer cover and into the absorbent assembly can improve the fading or disappearing quality of the active graphics. To enhance this effect, the outer cover can be attached to the absorbent assembly in a windowpane design, whereby the active graphic region of the outer cover is not bonded to the absorbent assembly and the regions of the outer cover surrounding the active graphic region are bonded to the absorbent assembly. One suitable method and apparatus for adhesively bonding the outer cover to the absorbent assembly in a windowpane design is disclosed in U.S. Pat. No. 5,683,752 issued Nov. 4, 1997 to Popp et al., which is incorporated herein by reference.

Additional Features which may be Used in Conjunction with the Present Invention:

A. Fastening System:

As noted previously, the illustrated training pant 20 has a side panel 34 disposed on each side of the absorbent chassis 32. In the illustrated embodiment, the pair of transversely opposed side panels 34 is permanently bonded to the absorbent chassis 32 and may be permanently bonded to one another, using attachment means known to those skilled in the art such as adhesive, thermal or ultrasonic bonding. Such a design allows the article to be used as a training pant. Training pants (also referred in the art as “pull-on” products) are placed in position on the wearer by inserting the wearer's legs into the leg openings and sliding the training pant into position about the wearer's lower torso. Suitable training pants are disclosed in U.S. Pat. No. 5,246,433, issued to Hasse, et al. on Sep. 21, 1993, U.S. Pat. No. 5,569,234, issued to Buell et al. on Oct. 29, 1996, U.S. Pat. No. 6,120,487, issued to Ashton on Sep. 19, 2000, U.S. Pat. No. 6,120,489, issued to Johnson et al. On Sep. 19, 2000, U.S. Pat. No. 4,940,464, issued to Van Gompel et al. on Jul. 10, 1990 and U.S. Pat. No. 5,092,861, issued to Nomura et al. on Mar. 3, 1992, the disclosure of each of which is incorporated herein by reference.

Generally, however, the diaper may include a variety of fastening systems. The fastening system preferably maintains the first waist region and the second waist region in a configuration so as to provide lateral tensions about the circumference of the diaper to hold the diaper on the wearer. The fastening system may comprise a surface fastener such as tape tabs, hook and loop fastening components and/or hermaphroditic fastening components, although any other known fastening means are generally acceptable. Some exemplary surface fastening systems are disclosed in U.S. Pat. No. 3,848,594 entitled “Tape Fastening System for Disposable Diaper” issued to Buell on Nov. 19, 1974; U.S. Pat. No. B 1 4,662,875 entitled “Absorbent Article” issued to Hirotsu et al. on May 5, 1987; U.S. Pat. No. 4,846,815 entitled “Disposable Diaper Having An Improved Fastening Device” issued to Scripps on Jul. 11, 1989; U.S. Pat. No. 4,894,060 entitled “Disposable Diaper With Improved Hook Fastener Portion” issued to Nestegard on Jan. 16, 1990; U.S. Pat. No. 4,946,527 entitled “Pressure-Sensitive Adhesive Fastener And Method of Making Same” issued to Battrell on Aug. 7, 1990; the herein before referenced U.S. Pat. No. 5,151,092 issued to Buell on Sep. 9, 1992; and U.S. Pat. No. 5,221,274 issued to Buell on Jun. 22, 1993. An exemplary interlocking fastening system is disclosed in co-pending U.S. application Ser. No. 09/143,184 entitled “Absorbent Article Fastening Device” in the names of Kline et al. filed on Aug. 8, 1998. The fastening system may also provide a means for holding the article in a disposal configuration as disclosed in U.S. Pat. No. 4,963,140 issued to Robertson et al. on Oct. 16, 1990. The fastening system may also include primary and secondary fastening systems, as disclosed in U.S. Pat. No. 4,699,622 entitled “Disposable Diaper Having An Improved Side Closure” issued to Toussant et al. on Oct. 13, 1987. to reduce shifting of overlapped portions or to improve fit as disclosed in U.S. Pat. No. 5,242,436 entitled “Absorbent Article With Fastening System Providing Dynamic Elasticized Waistband Fit” issued to Weil et al. on Sep. 7, 1993; U.S. Pat. No. 5,499,978 entitled “Absorbent Article With Dynamic Elastic Waist Feature Having A Predisposed Resilient Flexural Hinge” issued to Buell et al. on Mar. 19, 1996; U.S. Pat. No. 5,507,736 entitled “Absorbent Article With Dynamic Elastic Waist Feature Comprising An Expansive Tummy Panel” issued to Clear et al. on Apr. 16, 1996; U.S. Pat. No. 5,591,152 entitled “Absorbent Article With Dynamic Elastic Waist Feature Having A Predisposed Resilient Flexural Hinge” issued to Buell et al. on Jan. 7, 1997.

B. Containment Flaps:

To further enhance containment and/or absorption of body exudates, the training pant 20 can include a containment flaps to provide a barrier to the transverse flow of body exudates. Suitable constructions and arrangements for the containment flaps are generally well known to those skilled in the art and are described in U.S. Pat. No. 4,704,116 which issued Nov. 3, 1987 to Enloe; U.S. Pat. No. 4,808,178 which issued on Feb. 28, 1989 to Aziz et al; U.S. Pat. No. 4,909,803 which issued Mar. 20, 1990 to Aziz et al; U.S. Pat. No. 3,860,003 which issued on Jan. 14, 1975 to Buell; U.S. Pat. No. 4,695,278 which issued on Sep. 22, 1987 to Lawson; U.S. Pat. No. 4,795,454 which issued on Jan. 3, 1989 to Dragoo.

Additionally, an exemplary, non-limiting manner in which said containment flaps may be attached to the chassis is disclosed in U.S. patent application Ser. No. 10/434,378. Lastly, an exemplary, non-limiting manner for constructing said containment flap (e.g., made of a continuous cuff material; constructed with the use of a single continuous bond, etc.) is disclosed in U.S. patent application Ser. Nos. 10/824,121 and 10/824,122.

C. Elastics:

To further enhance containment and/or absorption of body exudates, the training pant 20 can include a front waist elastic member, a rear waist elastic member, and leg elastic members (not shown), as are known to those skilled in the art. Waist elastic members and leg elastic members can be operatively joined to the outer cover 40 and/or bodyside liner 42 of the training pant 20. Elastic members for the containment flaps, waist elastics and leg elastics can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic constrictive forces are imparted to the substrate. In one particular embodiment, for example, the leg elastic members comprise a plurality of dry-spun coalesced multifilament spandex elastomeric threads sold under the trade name LYCRA[R] and available from E. I. Du Pont de Nemours and Company, Wilmington, Del. In another embodiment, suitable elastics are strands having a cross section of 0.18 mm by 1.5 mm and made from natural rubber as available from Easthampton Rubber Company of Stewart, Va., under the trademark L-1900 Rubber Compound. Other suitable elastics can be made from natural rubber, such as elastic tape sold under the trademark Fulflex 9411 by Fulflex Company of Middletown, R. I. Elastics may also comprise any heat shrinkable elastic material as is well known in the art. Other suitable elastic materials may comprise a wide variety of materials as are well known in the art include elastomeric films, polyurethane films, elastomeric foams, formed elastic scrim and synthetic elastomers (e.g., Lycra™). In addition, elastics may take a multitude of configurations. For example, the width may be varied; a single strand or several parallel or non-parallel strands of elastic material may be used; or a variety of shaped may be used including rectilinear and curvilinear.

D. Sensors:

Biosensors may be used to detect a variety of characteristics (e.g., temperature, wetness, presence of a particular substance). One such example includes the use of a feel-wet liner or cool-feel liner. Feel-wet liners and other like products are described in U.S. Pat. No. 6,642,427, U.S. Pat. No. 6,627,786, U.S. Patent Application Publication No. 2003/0199845A1, and U.S. patent application Ser. No. 10/697,225.

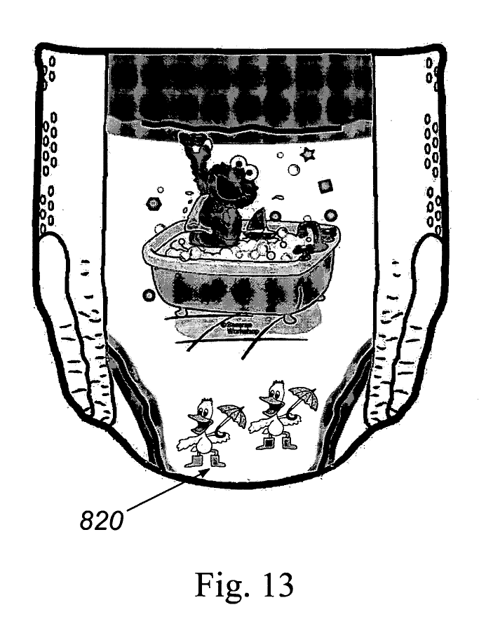

In fact, it is herein contemplated that feel-wet indications may be used in conjunction with active character graphics which themselves communicate the triggered response. For example, when the child feels wet from the feel-wet liner, the active character graphic 820 may be in the form of a rubber-duck having anthropomorphic features (e.g., smiling face, holding an umbrella, and wearing rubber boots; see FIG. 13). The active character graphic could also become/change to a particular color to denote a particular triggered response (yellow for detection of urine without fecal matter; brown for detection of fecal matter).

Other types of sensors include dehydration indicators, dryness indicators, biosensor for microbes, and skin health sensor.

Additionally, alarms (e.g., audible) may be used to further communicate the triggered response.

E. Lotions:

Lotions or other known substances may be used to add, enhance or change the performance or other characteristics of various parts of the training pant. For example, any portion of the topsheet may be coated with a lotion as is known in the art. Examples of suitable lotions include those described in U.S. Pat. No. 5,607,760 entitled “Disposable Absorbent Article Having A Lotioned Topsheet Containing an Emollient and a Polyol Polyester Immobilizing Agent” issued to Roe on Mar. 4, 1997; U.S. Pat. No. 5,609,587 entitled “Diaper Having A Lotion Topsheet Comprising A Liquid Polyol Polyester Emollient And An Immobilizing Agent” issued to Roe on Mar. 11, 1997; U.S. Pat. No. 5,635,191 entitled “Diaper Having A Lotioned Topsheet Containing A Polysiloxane Emollient” issued to Roe et al. on Jun. 3, 1997; U.S. Pat. No. 5,643,588 entitled “Diaper Having A Lotioned Topsheet” issued to Roe et al. on Jul. 1, 1997; U.S. Pat. No. 5,968,025 entitled “Absorbent Article Having a Lotioned Topsheet” issued to Roe et al. on Oct. 19, 1999 and U.S. Pat. No. 6,716,441 entitled “Compositions for the efficient release of active ingredients” issued to Osborne on Apr. 6, 2004. The lotion may function alone or in combination with another agent as the hydrophobizing treatment described above. The topsheet may also include or be treated with antibacterial agents, some examples of which are disclosed in PCT Publication No. WO 95/24173 entitled “Absorbent Articles Containing Antibacterial Agents in the Topsheet For Odor Control” which was published on Sep. 14, 1995 in the name of Theresa Johnson. Further, the topsheet, the backsheet or any portion of the topsheet or backsheet may be embossed and/or matte finished to provide a more cloth like appearance

All documents cited are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

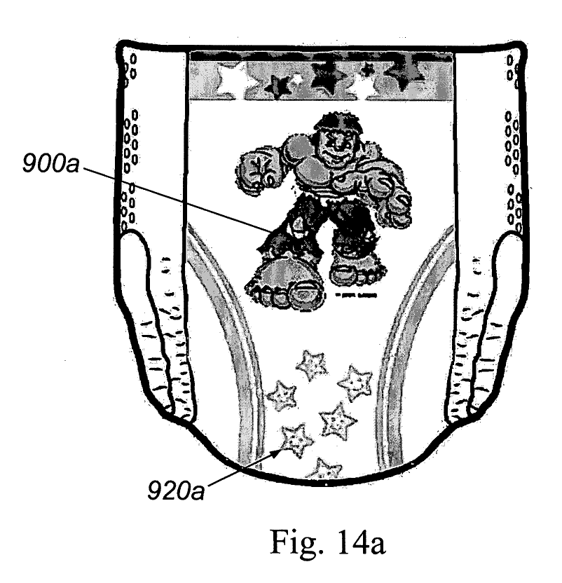

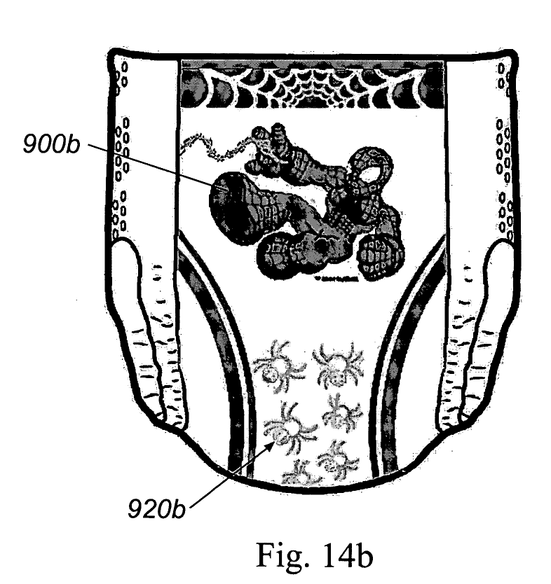

For example, FIGS. 14a and 14b depict additional exemplary, non-limiting embodiments of the present invention wherein the toilet training storyline is communicated with a permanent character graphic and an active character graphic without the presence of an object graphic. More specifically, a permanent character graphic 900a, 900b (see FIGS. 14a and 14b, respectively) is related in subject matter to an active character graphic 920a, 920b, respectively. In this way, an effective yet cheaper diaper may be provided (e.g., less ink) and even perhaps an easier-to-understand toilet training diaper may be provided for younger children (e.g., fewer number of graphics to explain to the younger child during the toilet training process). Consequently, one skilled in the art would appreciate that so long as an active character graphic is provided in combination with a permanent graphic (either character or object), an effective toilet training diaper may be provided.

What is claimed is:

1. An absorbent article comprising: an outer cover having an interior surface and an opposite exterior surface; an absorbent assembly disposed on the interior surface; a permanent character graphic disposed on the outer cover; a permanent object graphic disposed on the outer cover; wherein the permanent character graphic is interactively interrelated with the permanent object graphic; and further comprising at least one active character graphic, wherein said active character graphic is related in subject matter with said permanent character graphic and said permanent object graphic.

2. The absorbent article of claim 1, wherein the permanent character graphic is illustrated to be involved in an activity utilizing the permanent object graphics, and the permanent object graphics are the subject of the activity.

3. The absorbent article of claim 1, wherein the active character graphic becomes significantly more visible when exposed to urine.

4. The absorbent article of claim 1, wherein the active character graphic becomes significantly more visible with the passage of time when exposed to the environment but not exposed to urine.

5. The absorbent article of claim 1, wherein the active character graphic becomes significantly less visible when exposed to urine.

6. The absorbent article of claim 1, wherein the active character graphic becomes significantly less visible with the passage of time when exposed to the environment but not exposed to urine.

7. The absorbent article of claim 1, further comprising a feel-wet liner, said feel-wet liner provides a feel-wet indication to the wearer or caregiver, said active character graphic further visually communicates said feel-wet indication.

8. The absorbent article of claim 7, wherein said active character graphic changes to a different color to denote a particular triggered response.

9. The absorbent article of claim 1, wherein the active character graphic is selected from the group consisting of alphabet-friends, bubble-friends, musical-note-friends, paint-bucket-friends, pencil-friends, gift-friends, geography-book-friends, and rubber-duck holding an umbrella.

10. The absorbent article of claim 1, further comprising highlighting graphics selected from the group consisting of leg openings highlighting graphics, waist opening highlighting graphics, elastic leg band highlighting graphics, elastic waistbands highlighting graphics; product label highlighting graphics, written instructions and combinations thereof.

11. An absorbent article comprising: an outer cover having an interior surface and an opposite exterior surface; an absorbent assembly disposed on the interior surface; a permanent graphic disposed on the outer cover; and at least one active character graphic, wherein said active character graphic is related in subject matter with said permanent graphic.

12. The absorbent article of claim 11, wherein the permanent character graphic is a permanent character graphic.

13. The absorbent article of claim 11, wherein the permanent character graphic is a permanent object graphic.
